(19) United States
(12) Patent Application Publication
(10) Pub. No.: US 2023/0042559 A1
(43) Pub. Date: Feb. 9, 2023

(54) BONE ANCHOR

(71) Applicant: New York Society for the Relief of the Ruptured and Crippled, maintaining the Hospital for Special S, New York, NY (US)

(72) Inventor: Albert Diaz, Austin, TX (US)

(73) Assignee: New York Society for the Relief of the Ruptured and Crippled, maintaining the Hospital for Special S, New York, NY (US)

(21) Appl. No.: 17/792,780

(22) PCT Filed: Jan. 25, 2021
(86) PCT No.: PCT/US2021/013691

§ 371 Date: Jul. 14, 2022

Related U.S. Application Data
(60) Provisional application No. 62/962,289, filed on Jan. 17, 2020.
(60) Provisional application No. 63/049,494, filed on Jul. 8, 2020.

(51) Int. Cl. A61F 2/08

(52) U.S. Cl. CPC A61F 2/0811; A61F 2002/0841; A61F 2002/0852; A61F 2002/0888

(57) ABSTRACT

A bone anchor for coupling tissue to bone comprising an anchor body having a hollow interior, an open proximal end in fluid communication with the hollow interior, and a distal end; and a suture anchor extending from the anchor body and distal to the open proximal end.

### BACKGROUND OF THE DISCLOSURE

The exemplary embodiments of present invention relate generally to a bone anchor and, more specifically, to a bone anchor for coupling tissue, such as soft tissue including tendons and ligaments, or autografts or allografts with boney plugs, to bone as part of a reconstructive or other surgery.

Anchoring devices currently used, for example, for tenodesis can rely on an on-lay technique that can suffer from a lack of general applicability, may provide subpar healing, may not adequately protect the repair, and require that the tissue lie flat on the bone. Further, such on-lay techniques do not provide the security or satisfaction of docking the graft within a bony socket.

Other techniques to couple tissue to bone require the insertion of an interference screw, or other anchor within the same bony socket as the graft, or the deployment of a fixation button outside the far cortex of the bone. These techniques can present shortcomings, such as spinning of the graft, or damage to the graft, upon inserting the interference screw, poor purchase or bite, difficulty with visualization, and/or the need to deploy a separate anchor far removed from the graft site after drilling a long guide pin through bone and soft tissue.

Accordingly, there remains a need for devices and methods that overcomes the shortfalls of conventional anchors and allows a surgeon to efficiently and reliably affix and tension tissue within a bony socket.

### SUMMARY OF THE DISCLOSURE

A bone anchor for coupling tissue to bone comprising: an anchor body having: a hollow interior, an open proximal end in fluid communication with the hollow interior, and a distal end in fluid communication with the hollow interior; and a suture anchor extending from the anchor body and distal to the open proximal end.

In one embodiment, the anchor body includes a plurality of windows in fluid communication with the hollow interior. In one embodiment, the anchor body includes plurality of longitudinal slats, optionally further comprising one or more transverse slats extending orthogonally from the longitudinal slats. In one embodiment, the anchor body includes one or more bone engagement barbs or protrusions about an exterior of the anchor body. In one embodiment, the open proximal end is defined by an annular ring, which can optionally include one or more through holes.

In one embodiment, the anchor body includes a plurality of wings extending proximally from the open proximal end. In one embodiment, the proximal ends of the plurality of wings each extend outwardly at an angle from a longitudinal axis of the anchor body. In one embodiment, the plurality of wings are deformable and/or defined by a longitudinal slat.

In one embodiment, the anchor body's hollow interior has a diameter sized to receive and retain a soft tissue therein. In one embodiment, the anchor body's hollow interior is sized to form an interference fit with the soft tissue.

In one embodiment, the suture anchor comprises a saddle. In one embodiment, the suture anchor comprises a first through hole and a second through hole spaced from the first through hole.

In one embodiment, the suture anchor further comprises a strut link. In one embodiment, the saddle extends transversely across the anchor body to define a pair of openings. In one embodiment, the saddle extends adjacent the distal end of the anchor body. In one embodiment, the saddle traverses an annular ring of the suture anchor.

In one embodiment, the suture anchor comprises an eyelet, such as an eyelet adjacent to the distal end of the anchor body.

In one embodiment, the bone anchor is further provided with a shuttle for engagement with the bone anchor. In one embodiment, the shuttle is engaged with the bone anchor at a location between a first end adapted for connection to a suture tail and a second end.

Yet another embodiment of the subject disclosure provides a method for coupling tissue to bone that includes preparing a bony socket within a bone; introducing a bone anchor into the bony socket, the bone anchor having an open proximal end and a hollow interior extending unobturated from the opening and adapted to house tissue therein; a suture anchor in communication with the hollow interior; the suture anchor structured to receive and apply traction to a suture tail; and a shuttle capable of being placed in communication with the suture tail, engaged with the suture anchor. The method further includes coupling the suture tail with the shuttle; and applying traction to the shuttle or the suture tail through the suture anchor such that the soft tissue is secured and housed within the hollow interior of the bone anchor. In certain embodiments, an end of the soft tissue is positioned within a tension zone along a longitudinal length of the bone anchor.

In certain exemplary embodiments, the method further includes applying traction to the suture tail to increase the tension of the housed soft tissue; and applying traction to the housed soft tissue to decrease the tension of the housed soft tissue. The method can further include suturing the suture tail to secure the suture tail upon obtaining a desired tension of the soft tissue. The soft tissue can be, for example, a tendon, ligament or autograft or an allograft having a bony plug.

### BRIEF DESCRIPTION OF THE SEVERAL VIEWS OF THE DRAWINGS

The following detailed description of the exemplary embodiments of the subject disclosure will be better understood when read in conjunction with the appended drawings. For the purpose of illustrating the present disclosure, there is shown in the drawings exemplary embodiments. It should be understood, however, that the subject application is not limited to the precise arrangements and instrumentalities shown.

FIGS. 1A, 1B and 1C are elevation, cross-sectional, and plan views of a bone anchor in accordance with an exemplary embodiment of the subject disclosure;

FIGS. 1D, 1E and 1F are elevation, perspective, and plan views of a bone anchor in accordance with another exemplary embodiment of the subject disclosure;

FIG. 1G is and elevation view of a bone anchor in accordance with another exemplary embodiment of the subject disclosure;

FIG. 2 is a plan view of a bone anchor in accordance with another exemplary embodiment of the subject disclosure;

FIGS. 3A, 3B and 3C are elevation, perspective, and plan views of a bone anchor in accordance with another exemplary embodiment of the subject disclosure;

FIG. 4 is a plan view of a bone anchor in accordance with another exemplary embodiment of the subject disclosure;

FIGS. 5A and 5B are perspective and plan views of a bone anchor in accordance with another exemplary embodiment of the subject disclosure;

FIGS. 6A and 6B are perspective and plan views of a bone anchor in accordance with another exemplary embodiment of the subject disclosure;

FIG. 7 is a perspective view of a bone anchor in accordance with another exemplary embodiment of the subject disclosure;

FIG. 8 is a perspective view of a bone anchor in accordance with another exemplary embodiment of the subject disclosure;

FIGS. 9A, 9B, 9C, 9D and 9E are an elevation view, another elevation view, a top plan view, a bottom plan view and a perspective view of a bone anchor in accordance with another exemplary embodiment of the subject disclosure;

FIGS. 10A and 10B are views of the bone anchor of FIGS. 9A-9E being deployed and in use in a bony socket to dock a tissue;

FIGS. 11A-11E are perspective, elevation, another perspective, bottom plan, and longitudinal cross-sectional views of a bone anchor in accordance with another exemplary embodiment of the subject disclosure;

FIGS. 12A-12D are schematic views of a bone anchor according to the subject disclosure in operation receiving and housing a tendon; and

FIGS. 13A-13D are schematic views of the bone anchor of FIGS. 5A-5D in operation.

### DETAILED DESCRIPTION

Reference will now be made in detail to the exemplary embodiments of the subject disclosure illustrated in the accompanying drawings. Wherever possible, the same or like reference numbers will be used throughout the drawings to refer to the same or like features. It should be noted that the drawings are in simplified form and are not drawn to precise scale. In reference to the disclosure herein, for purposes of convenience and clarity only, directional terms such as upper, lower, top, bottom, above, below and diagonal, are used with respect to the accompanying drawings. Such directional terms used in conjunction with the following description of the drawings should not be construed to limit the scope of the subject disclosure in any manner not explicitly set forth. Additionally, the term “a,” as used in the specification, means “at least one.” The terminology includes the words above specifically mentioned, derivatives thereof, and words of similar import.

“About” as used herein when referring to a measurable value such as an amount, a temporal duration, and the like, is meant to encompass variations of ±20%, ±10%, ±5%, ±1%, or ±0.1% from the specified value, as such variations are appropriate.

“Substantially” as used herein shall mean considerable in extent, largely but not wholly that which is specified, or an appropriate variation therefrom as is acceptable within the field of art. “Exemplary” as used herein shall mean serving as an example.

Throughout the subject application, various aspects thereof can be presented in a range format. It should be understood that the description in range format is merely for convenience and brevity and should not be construed as an inflexible limitation on the scope of the subject disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Furthermore, the described features, advantages and characteristics of the exemplary embodiments of the subject disclosure may be combined in any suitable manner in one or more embodiments. One skilled in the relevant art will recognize, in light of the description herein, that the subject disclosure can be practiced without one or more of the specific features or advantages of a particular exemplary embodiment. In other instances, additional features and advantages may be recognized in certain embodiments that may not be present in all exemplary embodiments of the present disclosure.

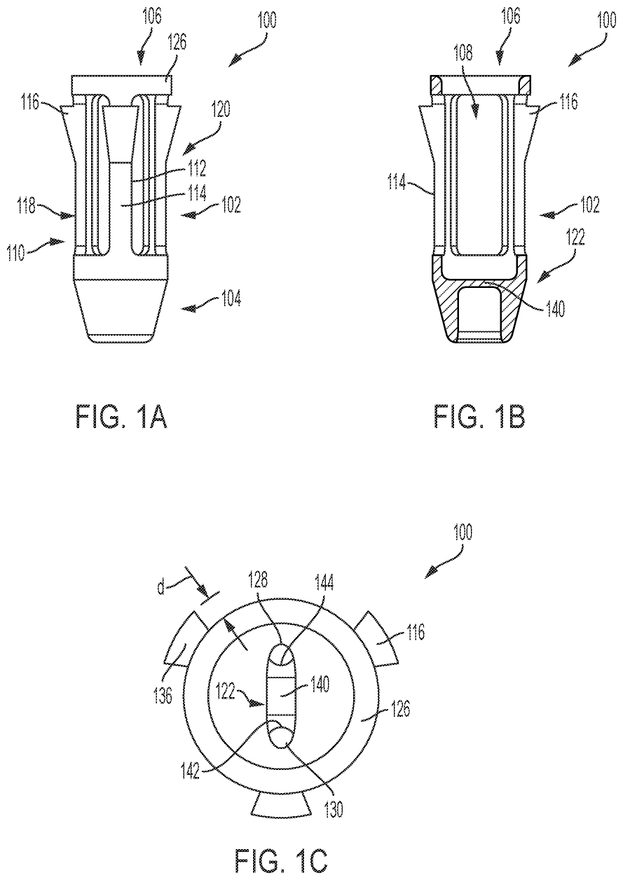

Referring now to the drawings, the Figures disclose bone anchors according to exemplary embodiments of the subject disclosure. With reference to FIGS. 1A-1C, the bone anchor 100 includes an anchor body 102 and a suture anchor 122 extending from the anchor body and distal to an open proximal end 106. The open proximal end 106 is in fluid communication (e.g., in unobturated fluid communication) with a hollow interior 108. The anchor body 102 further includes a distal end 110 that, in this non-limiting embodiment, is also in fluid communication with the hollow interior 108.

The anchor body 102 is tubular or substantially tubular along its longitudinal length, having a maximum inner diameter about the proximal end 106 that, in this exemplary embodiment, includes an annular ring 126. The anchor body 102 maintains this maximum inner diameter along a first section 120 that includes a plurality of windows 112 (e.g., three windows). The anchor body is provided with an open architecture frame without intervening obstacles between its proximal end 106 and distal end 110 as well as the suture anchor 122.

In certain non-limiting embodiments, the inner diameter of the bone anchor along the first section 120 can be sized to provide an interference fit with tissue that is to be docked within the bone anchor, as will be described in greater detail below. For example, for bicep tenodesis, the inner diameter of the anchor body (e.g., the diameter along first section 120) can be provided or sized such that the bicep tendon can be urged into the first section, and once introduced, will not move absent a force, such as a predetermined force greater than an interference fit force sufficient to secure the weight of the bone anchor to the tendon, being applied thereto. Accordingly, the inner diameter of the anchor body can, in certain exemplary embodiments, be selected based on the size of the soft tissue that is to be docked in the bone anchor.

The windows 112 in this exemplary embodiment are formed from (e.g., bounded by) three, equally spaced longitudinal slats 114 forming three windows. However, the bone anchor can be configured to have more or less than three windows e.g., 2, 4, 5, 6 or more windows formed from respective longitudinal slats, as will be further discussed below.

The longitudinal slats are each provided with one or more bone engagement barbs or protrusions 116 about an exterior 118 of the anchor body. The barbs or protrusions 116 include a lateral flare 136 extending an annular distance, d, from annular ring 126 and defining a maximum radial reach of the tubular body.

The suture anchor 122 extends from the body along a section 104 having e.g., a generally tapered configuration such as a frustrum shape. This section 104 is distal to the first section 120 and adjacent the distal end 110 of the anchor body. The suture anchor 122 in this exemplary embodiment is in the form of a saddle and includes a post 140 or other cross bar structure that allows for a suture to secured thereto in the manner as will be discussed below. The post 140 includes an anterior face 142, and a posterior face 144 each provided with a concave surface to direct a suture (or suture instrumentalities) towards the center of the first and second through holes 128, 130. A suture instrumentality can be received from the proximal end 106, received in first through hole 128, directed under post 140 and back up through the second through hole 130 and drawn back toward the proximal end 106.

Alternatively expressed, the suture anchor 122 in this exemplary embodiment includes a first through hole 128 and a second through hole 130 spaced from the first through hole, each through hole sized sufficiently to receive a suture or a suture instrumentality (e.g., a shuttle or tails from a suture). The first and second through holes 128, 130 are each in fluid communication with the hollow interior 108, and also in fluid communication with the distal end 110 of the anchor body. The post 140 is spaced a distance from the distal end of the bone anchor sufficient to pass sutures under the post 140 without contacting bone.

As shown in FIG. 1A, section 104 of bone anchor 100 has a solid circumferential exterior. Other configurations can alternatively be provided about the distal end of the bone anchor. For example, as shown in FIGS. 1D-1F in connection with bone anchor 100′ (which is similarly constructed to that of bone anchor 100) a pair of slots 124a′, 124b′ is provided along opposing lateral sides of section 104′ extending towards its distal end. The slots in combination with the post 140′ of the suture anchor 122′ (extending in a widthwise direction) define a third through hole 132′ and a channel 134′, each in fluid communication with the first and second through holes 128′, 130′.

Alternatively, as shown in FIG. 1G in connection with bone anchor 100″ in accordance with another exemplary embodiment of the subject disclosure and similarly constructed to bone anchor 100′ except as discussed, slots 124″ can terminate at an inferior border 135″ that circumscribes the body and yields a fourth through hole 134″ (in place of channel 134′ of bone anchor 100′). The fourth through hole 134″ also being in fluid communication with first and the second through holes (similar to first and second through holes 128′, 130; of embodiment 100′) and spaced from third through hole 132″.

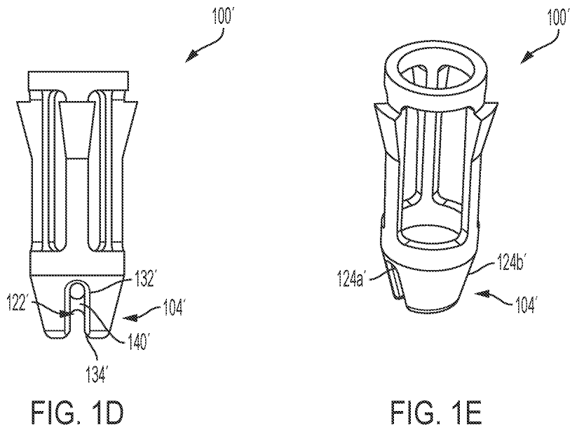

As shown in FIG. 2, alternative embodiments of a bone anchor according to the subject disclosure can be provided with different flare widths and designs, such as shown for bone anchor 200 that is provided with bone engagement barbs or protrusions 216 with lateral flares 236 having an increased flare width d′ (all other features of the bone anchor being similar to or applicable to the features of the other embodiments of the bone anchors described herein). The width of the flare can be varied as necessary to provide sufficient resistance to the bone anchor from being pulled out of a bony socket.

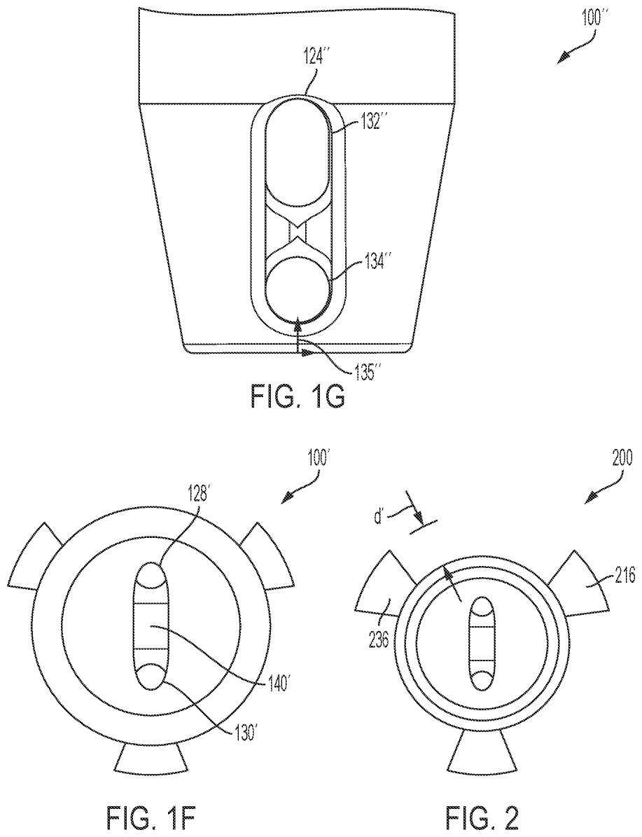

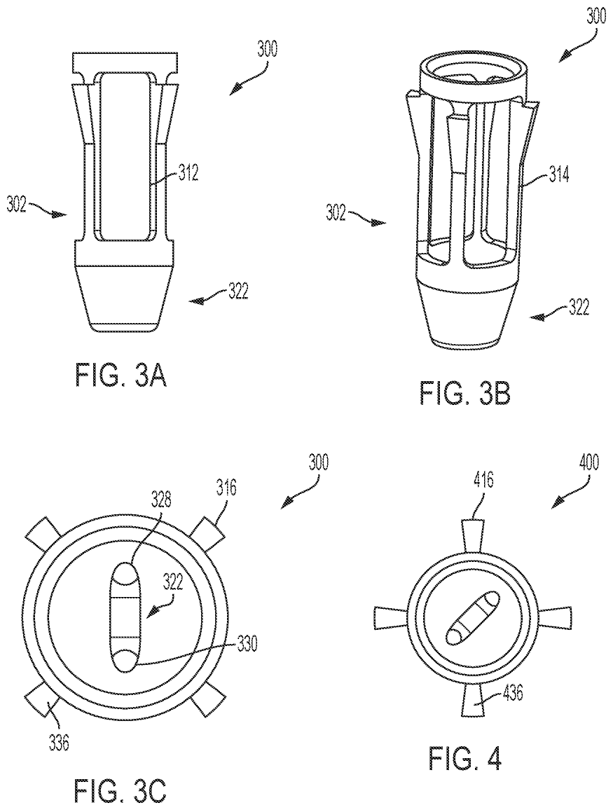

Further, other configurations can alternatively be provided in accordance with the subject disclosure, such as bone anchor 300 shown in FIGS. 3A and 3B in which the body 302 includes four, equally spaced longitudinal slats 314 in turn providing four windows 312 in like manner as anchor 100. Bone anchor 300 is otherwise similar to bone anchors 100 and 200, and includes a suture anchor 322 in the form of a saddle having a first through hole 328 and a second through hole 330 and bone engagement barbs or protrusions 316 having a flare 336. As shown in FIG. 4, alternative embodiments, such as bone anchor 400, are provided with bone engagement barbs or protrusions 416 having a flare 436 with an increased width, as compared, for example, to flare 336 of bone anchor 300.

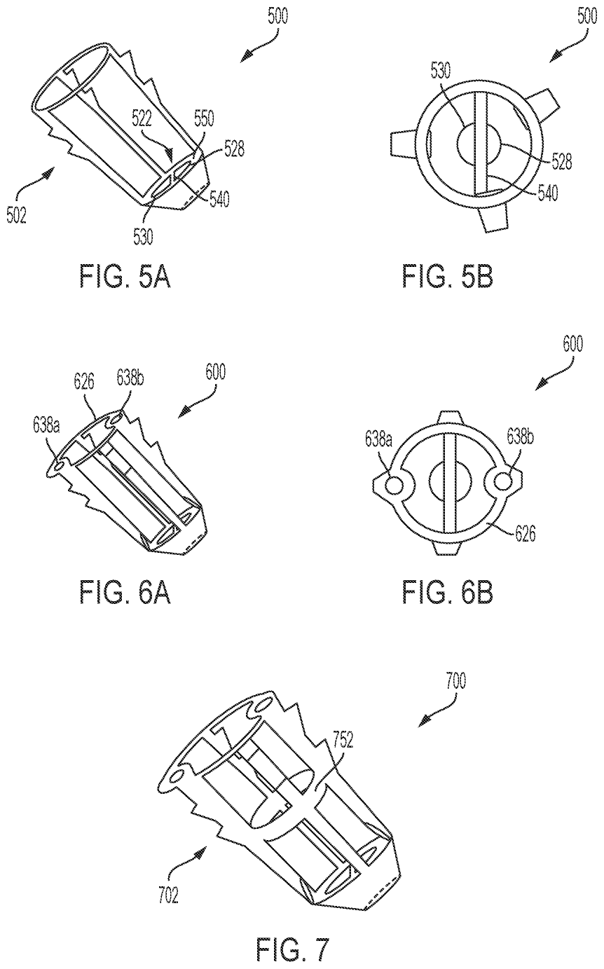

FIGS. 5A-5B illustrate bone anchor 500 according to an alternative exemplary embodiment of subject disclosure having a suture anchor 522 that includes a post 540 that is in the form of a strut link or like member. The strut link 540 extends transversely across an annular ring 550 of the suture anchor and extends across the width of the anchor body to define a first through hole 528 and a second through hole 530 spaced from the first through hole.

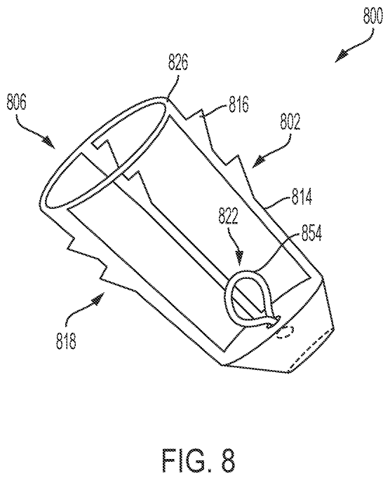

FIGS. 6A and 6B illustrate another exemplary embodiment of a bone anchor 600 according to the subject disclosure. The bone anchor 600 includes an annular ring 626 and can optionally include one or more through holes 638a, 638b. Through holes 638a, 638b can be provided to assist in final or supplementary suturing procedures once the soft tissue is docked within the bone anchor and the desired tension is achieved via manipulation of the soft tissue within a tension or tensioning zone, as will be discussed below. Through holes 638a and 638b are not required and optionally included in certain non-limiting embodiments.

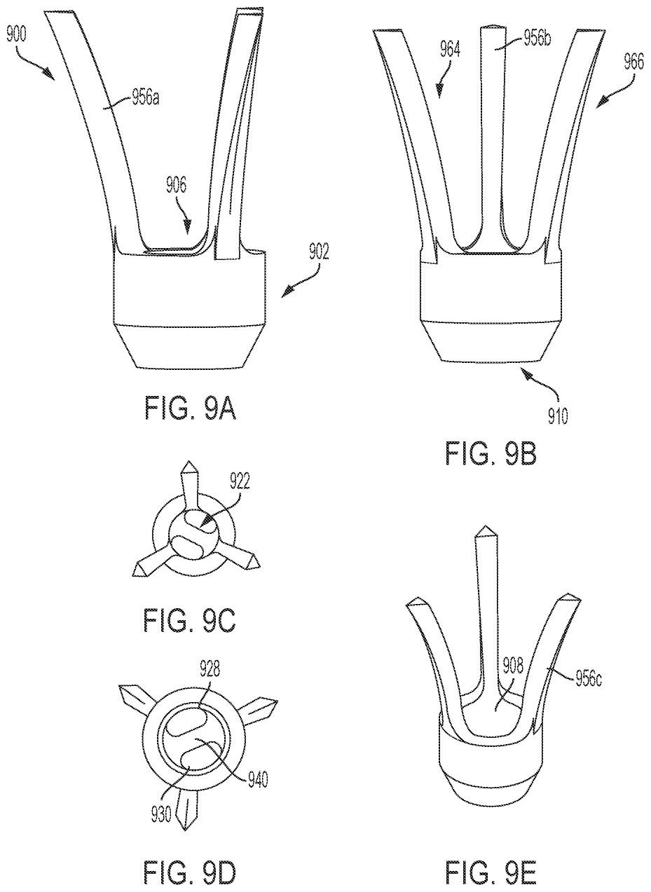

FIG. 7 discloses bone anchor 700 according to another exemplary embodiment of the subject disclosure. Bone anchor 700 includes an anchor body 702 that includes a plurality of transverse slats 752 that, in this exemplary embodiment, are joined to circumscribe the anchor body 702 to form a web-like or cage design. The subject disclosure is not limited to this exemplary embodiment, as other variations with additional transverse slats or other orthogonal or diagonal or other configurations of slats can be utilized to form a cage design.

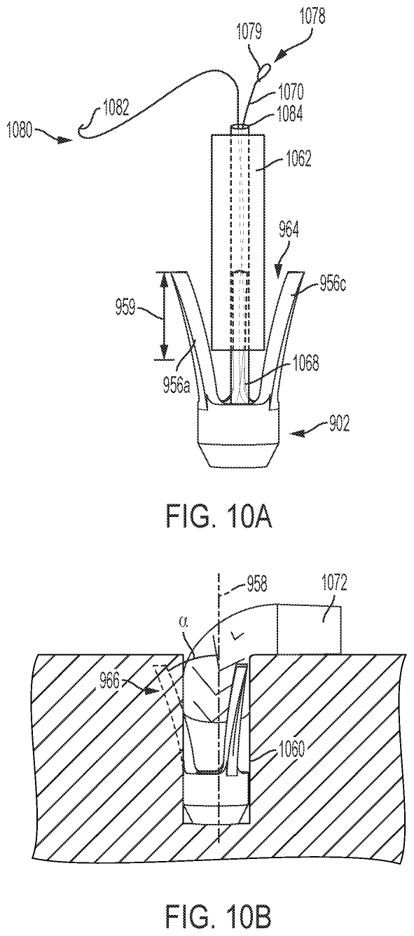

The suture anchor of the present invention is further not limited to the saddle configurations disclosed in the above exemplary embodiments. For example, FIG. 8 illustrates another exemplary embodiment of a bone anchor 800 having a suture anchor 822 that includes an eyelet 854 extending in the proximal direction. Bone anchor 800 can otherwise be similarly structured to bone anchor 100 and includes a proximal end 806 defined by an annular ring 826, longitudinal slats 814 extending therefrom provided with bone engagement barbs or protrusions 816 extending about an exterior 818 of the anchor body 802.

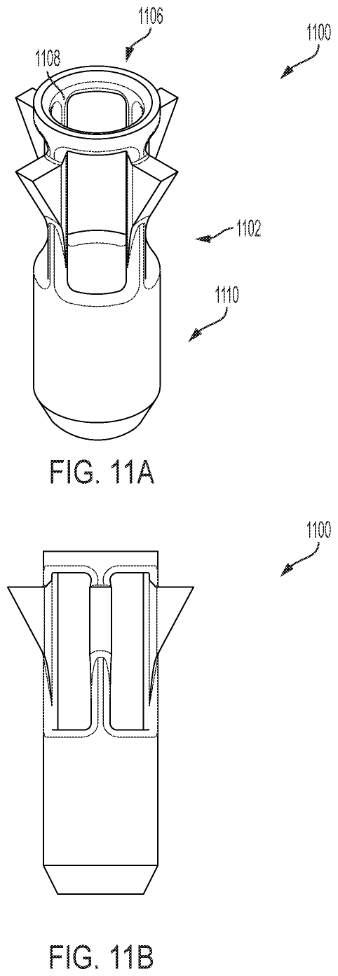

FIGS. 9A-9E illustrate bone anchor 900 according to another exemplary embodiment of the subject disclosure. The bone anchor 900 includes an anchor body 902 having a hollow interior 908, an open proximal end 906 in fluid communication with the hollow interior, and a distal end 910. The distal end is in fluid communication with the hollow interior. The bone anchor 900 further includes a suture anchor 922 in the form of a saddle adjacent the distal end 910 of the anchor body 902 and distal to the open proximal end 906.

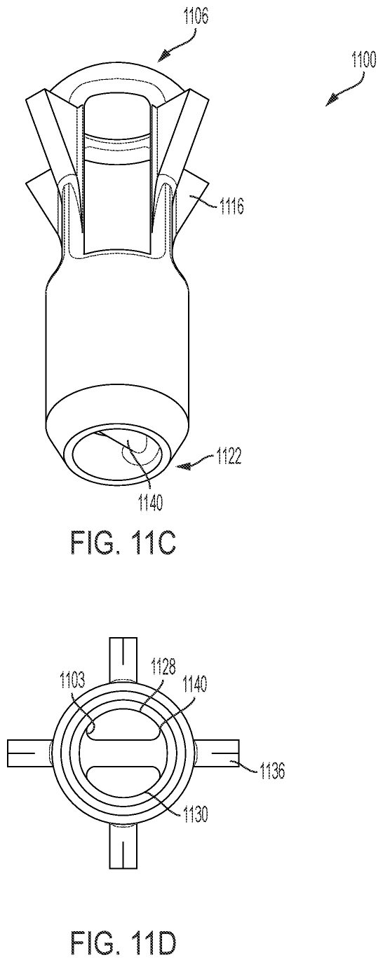

The bone anchor 900 includes a plurality of wings 956a, 956b, 956c extending proximally from the open proximal end 906. In this embodiment, three wings are provided, although any number of wings (e.g., 2 wings, 4 wings or 5 wings or more) can alternatively be provided. The proximal end 966 of the wings 956a, 956b, 956c extend outwardly e.g., at an oblique angle, a, from a central longitudinal axis 958 of the anchor body (FIG. 10B). Each of the plurality of wings are elongated slats or finger-like projections, that in this exemplary embodiment have a triangular longitudinal cross-sectional shape, which define radially extending edges to assist engagement with the bony socket. Alternatively, other cross-sectional shapes can be employed with or without edges.

The saddle 922 is similar to saddle 122 and includes a post 940 defining a first through hole 928 and a second through hole 930 spaced from the first through hole. However, that structure of the suture anchor is not limited to that shown in this exemplary embodiment, but may alternatively be configured as any other saddle or strut of any of the other embodiments of the present disclosure or any other cross bar or eyelet suitable for its intended purpose.

Each of the plurality of wings 956a, 956b, 956c are deformable. With reference to FIGS. 10A and 10B, the wings of bone anchor 900 can deform to allow it to be inserted into a bony socket 1060. For example, the plurality of wings can move between an extended or first position (FIG. 9A) and a contracted or second position wherein the plurality of wings is biased inwardly to a fit within a counterbore in bone. That is, the plurality of wings can be biased inwardly such that the overall diameter of the wings is substantially equal to an overall diameter of the anchor body 902. Additionally, the plurality of wings are biased to be in the extended or first position as shown in FIG. 9A.

With reference to FIG. 10A, an implant delivery system, such as a dilator 1062 can be provided with a cannulated inserter 1068 for inserting the bone anchor into bone. In operation, the dilator surrounds a suture shuttle 1070 mounted therein as shown in FIG. 10A. The implant delivery system can further include a handle 1084 to secure the suture shuttle 1070 thereto.

The suture shuttle is a device that is used to pass a suture through the bone anchor and includes a first end 1078 with a loop 1079 or mechanism adapted to be connected to a suture tail and a second end 1080 containing a tip 1082.

In operation, the dilator 1062 is introduced into an interior 964 defined by the plurality of wings to apply a force to expand a proximal portion 966 of the wings into bone after the bone anchor has been inserted into a bone socket or counterbore, as shown in FIG. 10B. Once dilated, the bone anchor 900 provides a zone 959 to house soft tissue within the interior 964 in which there, beneficially, is no implant between the housed tissue 1072 and the bony socket 1060, thereby providing a tighter fit of the soft tissue within the bony socket and obviating the need to increase the size of the bony socket in order to accommodate the bone anchor. Accordingly, bone anchor 900 allows for smaller socket sizes to be provided relative to the graft diameter. Additionally, the bone anchor 900 as well as the other bone anchors of the subject disclosure advantageously allow for more surface area exposure of the tissue or tendon to bone (e.g., via windows) which allow for better healing and less damage to the tissue and/or tendon.

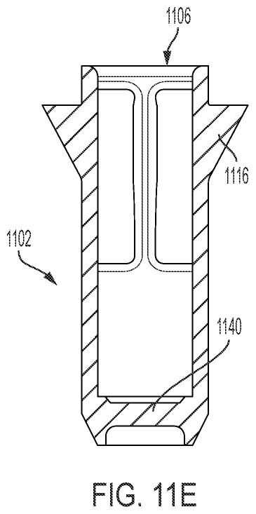

FIGS. 11A-11E illustrate bone anchor 1100 according to another exemplary embodiment of the subject disclosure. The bone anchor 1100 includes an anchor body 1102 having a hollow interior 1108, an open proximal end 1106 in fluid communication with the hollow interior, and a distal end 1110. The distal end is in fluid communication with the hollow interior. The bone anchor 1100 further includes a suture anchor 1122 in the form of a saddle adjacent the distal end 1110 of the anchor body 1102 and distal to the open proximal end 1106.

The anchor body 1102 of bone anchor 1100 is provided with a plurality of longitudinal slats 1114 that define an equal number of windows 1112. Each slat 1114 includes a bone engagement barb or protrusion 1116 that include a lateral flare 1136 or a lateral flare of variable lengths, as discussed above.

The suture anchor 1122 is similar in structure to suture anchor 122, discussed above, and is likewise in the form of a saddle and includes a post 1140 or other cross bar structure that allows for a suture to be secured thereto. The post 1140 is in the form of an annular or rounded cross bar extending across the anchor body 1102, with outward flares 1103 provided about the ends of the post where it engages from the anchor body. A suture instrumentality can be received from the proximal end 1106, received in first through hole 1128, directed under post 1140 and back up through the second through hole 1130 and drawn back toward the proximal end 1106.

The bone anchor 1100 as well as the bone anchors of the other exemplary embodiments discussed above can be formed from any biocompatible material of sufficient strength. For example, materials commonly used in implants, such as metal and metal alloys (e.g., stainless steel, titanium, nitinol) or other materials known in the art can be employed in accordance with the disclosed subject matter. Additionally, the bone anchors can be composed of, or include one or more of, a radiolucent metal, a thermoplastic (e.g., PEEK), a bio-composite, and/or carbon fiber reinforced polymers, for example.

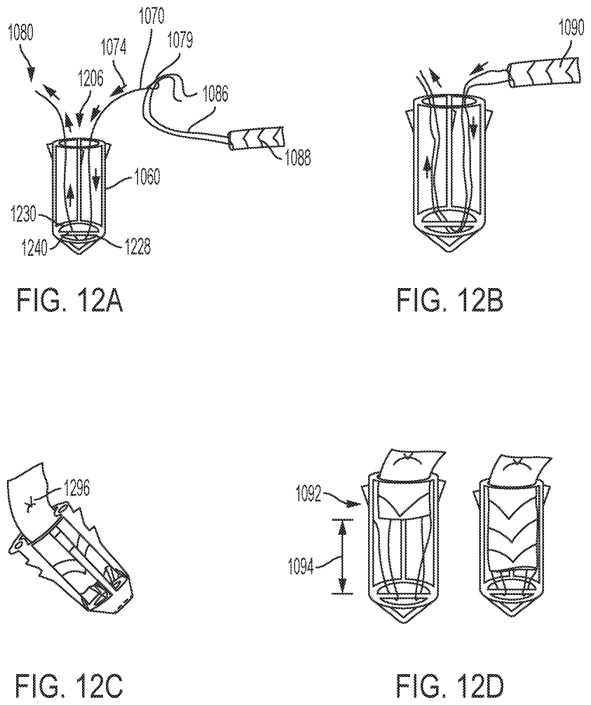

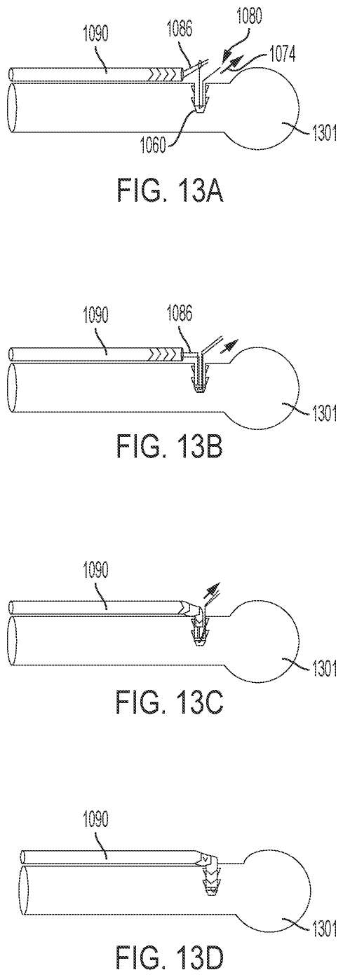

Operation of the bone anchors according to the subject disclosure will now be described in connection with FIGS. 12A-13D. Prior to being used, the bone anchor can be provided pre-loaded with a suture shuttle, such as suture shuttle 1070. More particularly, as removed from a sterile packaging, the second end 1080 of the suture shuttle 1070 can be pre-loaded into the proximal end 1206 of the bone anchor, received in a first through hole 1228, directed under post 1240 and then up through a second through hole 1230 and drawn back out the proximal end 1206 to provide the configuration shown in FIG. 12A.

Referring to FIGS. 12A-12D, the bony socket 1060 is prepared in a bone 1301 using a tool, for example, a straight awl or drill. Also, suture tails 1086 from a locking suture or whip stitch 1088 from a tendon 1090 is engaged with a loop 1079 of the suture shuttle 1070. A bone anchor e.g., any of the bone anchors described above, can then be inserted into the bony socket 1060 for example, by hand or with the use of a tool e.g., a mallet. Additionally, a dilator can be applied to deploy wings of an applicable bone anchor e.g., bone anchor 900, into the bone 1301 from within the bony socket 1060.

The second end 1080 of the suture shuttle is advanced in the directions of the arrows 1074 to shuttle the suture tails 1086 from the tendon 1090 through the bone anchor as shown in FIGS. 12A and 12B. Traction is continued to be applied to the suture tail 1086 until the implant is docked within the hollow interior of the bone anchor as shown in FIGS. 12C and 13C.

The foregoing bone anchors of the present disclosure advantageously allows a user to adjust the tension applied to the tissue or tendon attached to the bone anchor. This tension adjustability includes both increasing tension as well as decreasing the tension of the tissue or tendon secured to the bone anchor and docked within the hollow interior of the anchor body. For example, the tension of the tendon 1090 can be adjusted by manipulation of a distal end 1092 of the tendon within a tension zone 1094 along the length of the bone anchor. To provide more tension to the tendon 1090, traction is applied to suture tails 1086 thereby drawing a distal end 1092 of the tendon 1090 further within the hollow interior of the bone anchor. To provide less tension to the tendon, traction is applied to the docked tendon, thereby drawing the distal end 1092 of the soft tissue in a direction towards the proximal end 1006, but still within the tension zone 1094. Once the desired tension is obtained, the suture tails 1086 can be tied in a knot 1296 to finally secure the tendon 1090 in place. As will be appreciated, the length of tension zone 1094 can be increased by increasing the length of the bone anchor and thus, differing lengths of bone anchors can be provided based on the tension excursion requirements for the tendon or tissue at issue.

While docking a tendon is described above in connection with tenodesis, the bone anchors of the subject application can be used to dock any native or cadaveric soft tissue, such as ligaments. For example, the bone anchors of the subject disclosure can be used in connection with a proximal biceps tenodesis, MPFL reconstruction, and other reconstructive procedures where a graft is affixed to bone, including complex knee, foot and ankle reconstructions. By the same process, soft tissue autografts and allografts with bony plugs, such as patellar tendons or the Achilles can be docked with the bone anchors of the subject disclosure.

It will be appreciated by those skilled in the art that changes could be made to the exemplary embodiments described above without departing from the broad inventive concept thereof. It is to be understood, therefore, that this disclosure is not limited to the particular embodiments disclosed, but it is intended to cover modifications within the spirit and scope of the claims defined herein.

1. A bone anchor for coupling tissue to bone comprising:
an anchor body having:
a hollow interior,
an open proximal end in fluid communication with the hollow interior, and
a distal end in fluid communication with the hollow interior; and
a suture anchor extending from the anchor body and distal to the open proximal end.

2. The bone anchor of claim 1, wherein the anchor body includes a plurality of windows in fluid communication with the hollow interior, wherein each of the plurality of windows extends substantially an entire longitudinal length of the anchor body.

3. The bone anchor of claim 1, wherein the anchor body includes plurality of longitudinal slats, each between a pair of adjacent windows of the plurality of windows.

4. The bone anchor of claim 3, further comprising one or more transverse slats extending orthogonally from the longitudinal slats.

5. The bone anchor of claim 3, wherein the plurality of longitudinal slats includes one or more bone engagement barbs or protrusions about its exterior.

6. The bone anchor of claim 1, wherein the open proximal end is defined by an annular ring.

7. The bone anchor of claim 1, wherein the anchor body includes a plurality of wings extending proximally from the open proximal end.

8. The bone anchor of claim 7, wherein the proximal ends of the plurality of wings each extend outwardly at an angle from a longitudinal axis of the anchor body.

9. The bone anchor of claim 7, wherein the plurality of wings are deformable.

10. The bone anchor of claim 9, wherein each of the plurality of wings are defined by a longitudinal slat.

11. The bone anchor of claim 1, wherein the hollow interior has a diameter sized to receive and retain a soft tissue therein.

12. The bone anchor of claim 11, wherein the hollow interior is sized to form an interference fit with the soft tissue.

13. The bone anchor of claim 1, wherein the suture anchor comprises a saddle.

14. The bone anchor of claim 1, wherein the suture anchor comprises a first through hole and a second through hole spaced from the first through hole.

15. The bone anchor of claim 1, wherein the suture anchor comprises a strut link.

16. The bone anchor of claim 13, wherein the saddle extends transversely across the anchor body to define a pair of openings.

17. The bone anchor of claim 13, wherein the saddle extends adjacent the distal end of the anchor body.

18. The bone anchor of claim 13, wherein the saddle traverses an annular ring of the suture anchor.

19. The bone anchor of claim of claim 1, wherein the suture anchor comprises an eyelet.

20. The bone anchor of claim 19, wherein the eyelet is adjacent the distal end of the anchor body.

21. A bone anchor assembly comprising:
the bone anchor of claim 1; and
a shuttle for engagement with the bone anchor.

22. The bone anchor assembly of claim 19, wherein the shuttle is engaged with the bone anchor a location between a first end adapted for connection to a suture tail and a second end.

23. (canceled)

24. (canceled)

25. (canceled)

26. (canceled)

27. (canceled)

28. (canceled)

29. (canceled)

30. (canceled)

31. (canceled)

32. The bone anchor of claim 7, wherein each of the plurality of wings curve outwardly.

33. The bone anchor of claim 7, wherein at least one of the plurality of wings have a triangular longitudinal cross-sectional shape.

34. The bone anchor of claim 7, wherein each of the plurality of wings have a triangular longitudinal cross-sectional shape.
